(19) United States
(12) Patent Application Publication
(10) Pub. No.: US 2021/0101001 A1
(43) Pub. Date: Apr. 8, 2021

(54) METHOD OF FABRICATING AN ELECTRODE STRUCTURE

(71) Applicant: THE UNIVERSITY OF MELBOURNE, Melbourne (AU)

(72) Inventors: Nicholas APOLLO, Melbourne (AU); David GARRETT, Melbourne (AU)

(21) Appl. No.: 16/762,840

(22) PCT Filed: Nov. 9, 2018
(86) PCT No.: PCT/AU2018/051210

§ 371 Date: May 8, 2020

(30) Foreign Application Priority Data
Nov. 10, 2017 (AU) 2017904574

(51) Int. Cl. A61N 1/05; H01M 4/96; H01M 4/04; H05K 1/03; H05K 3/00

(52) U.S. Cl. CPC A61N 1/0534; H01M 4/96; H05K 3/0047; H05K 1/0313; H01M 4/04

(57) ABSTRACT

The present disclosure provides a method of fabricating an electrode structure. The method provides an electrically insulating substrate having a first surface, a second surface opposite the first surface, and a plurality of through-holes, each through-hole extending across a thickness of the insulating substrate. The method further comprises extruding a material sequentially or simultaneously through at least some of the through-holes resulting in a plurality of elongate electrically conductive elements extending through and protruding from the through-holes at the first surface of the electrically insulating substrate. In addition, the method comprises forming a plurality of electrically conductive regions at the second surface of the electrically insulating substrate. Each electrically conductive region is located at a respective through-hole, whereby the electrically conductive regions are electrically coupled to the elongate electrically conductive elements.

### TECHNICAL FIELD

The present disclosure relates to a method of fabricating an electrode structure.

### BACKGROUND ART

Medical devices for implantation into the human body, such as retinal prostheses or deep brain stimulators, typically have electrodes for transmitting electrical signals. The electrodes are used for establishing communication with neurons and need to be biocompatible to enable a successful integration of the medical implant within the human tissue.

Further, it is important that the electrodes have a small size and a high density of electrical contacts to enable contacting a large number of the relatively small neurons.

The publication by Nicholas V. Apollo et al., “Soft, flexible freestanding neural stimulation and recording electrodes fabricated from reduced graphene oxide” relates to the fabrication of a conductive micro-fibre for use in neural stimulation and recording electrodes.

### SUMMARY OF THE DISCLOSURE

The present invention provides a method of fabricating an electrode structure, the method comprising:

providing an electrically insulating substrate having a first surface, a second surface opposite the first surface, and a plurality of through-holes, each through-hole extending across a thickness of the insulating substrate;

extruding a material through at least some of the through-holes resulting in a plurality of elongate electrically conductive elements extending through and protruding from the through-holes at the first surface of the electrically insulating substrate; and

forming a plurality of electrically conductive regions at the second surface of the electrically insulating substrate, each electrically conductive region being located at a respective through-hole, whereby the electrically conductive regions are electrically coupled to the elongate electrically conductive elements.

Each elongate electrically conductive element may extend through and protrude from a respective through-hole.

The step of extruding a material through at least some of the through-holes may comprise extruding the material sequentially through at least some of the holes. Alternatively, the step of extruding a material through at least some of the through-holes may comprise extruding the material simultaneously through at least some of the holes.

The electrode structure comprises an array of electrodes. The array may comprise more than 20, more than 50, more than 100 or more than 500 electrodes and elongate electrically conductive elements per square millimetre. The material that is extruded through at least some of the through-holes may comprise carbon.

In a first embodiment of the present invention the step of extruding a material through at least some of the through-holes comprises moving the plurality of elongate electrically conductive elements through the through-holes. Each elongate electrically conductive element is typically moved through a respective through-hole and may be moved using a press, such as a hydraulic press.

In this embodiment, forming the plurality of electrically conductive regions at the second surface of the electrically insulating substrate comprises bonding the elongate electrically conductive elements to the substrate.

Forming the plurality of electrically conductive regions at the second surface of the electrically insulating substrate may further comprise sealing the elongate electrically conductive elements and the substrate. Sealing may comprise hermetic sealing.

In one embodiment, the elongate electrically conductive elements are carbon fibres or rods. Alternatively, the elongate electrically conductive elements may for example be metallic micro-wires.

The plurality of elongate electrically conductive elements that are moved through the through-holes may comprise at least 100, such as 100-500, 500-1000 or more than 1000 elongate electrically conductive elements which are electrically-insulated from one another and, therefore, acquire or deliver distinct signals.

In an alternative second embodiment of the present invention the step of extruding the material comprises extruding a liquid material or a paste through at least some of the through-holes, the material being selected and extruded such that a plurality of solid elongate electrically conductive elements are formed when the material has hardened.

The liquid material or paste may comprise a conductive ink. The conductive ink may comprise graphene.

Alternatively, the conductive ink may comprise a conductive polymer.

At least some of the elongate electrically conductive elements may have a substantially circular cross-sectional shape with a diameter in the range of 2-20 μm, such as 2-5 μm, 5-7 μm, 7-10 μm or greater than 10 μm.

The elongate electrically conductive elements may protrude from the through-holes at the first surface of the electrically insulating substrate with a length of at least 0.01 mm, at least 0.1 mm, such as between 1 mm and 10 mm.

In one embodiment, each through-hole has a tapered cross-sectional shape.

Providing the electrically insulating substrate may comprise drilling the through-holes into the electrically insulating substrate using an ablation technique such as laser ablation or using a focused ion beam.

Generally, the electrically insulating substrate may comprise a material that is biocompatible. The electrically insulating substrate may comprise a diamond material such as poly-crystalline or single-crystalline diamond material. Alternatively, the insulating substrate may comprise a ceramic material such as alumina, sapphire, and/or silicon carbide.

In one embodiment, the electrically conductive regions are formed using a brazing alloy paste.

A surface of the electrically conductive regions may project from the second surface of the electrically insulating substrate or may be flush with the second surface.

The electrically conductive regions may be arranged for mounting or bonding to an electronic component such as a microprocessor or an application specific circuit (ASIC).

In one embodiment, the method further comprises forming an electrically insulating layer on at least a portion of a surface of the elongate electrically conductive elements or on an entire exposed surface of the elongate electrically conductive elements to electrically insulate the electrically conductive elements from each other. The electrically insulating layer may be formed using a vacuum deposition technique and may comprise for example silicon dioxide and/or a poly(p-xylylene) polymer (parylene).

The method may further comprise forming an electrically conductive layer on a surface of distal end portions or tips of the elongate electrically conductive elements protruding from the through-holes. The electrically conductive layer may be suitable for improving a biocompatibility and/or electrochemical properties of the electrode structure.

The electrically conductive layer may be formed using an electrochemical deposition technique, and may comprise a conductive polymer, platinum group metals, or a doped electrically conductive diamond material, such as a boron or nitrogen doped diamond material.

Alternatively, the electrically conductive layer may comprise organic molecules suitable for electrochemically functionalizing the surface of the distal end portions or tips of the elongate electrically conductive elements.

Further, the method may comprise removing portions of the formed insulating layer from localised positions at side portions of the elongate electrically conductive elements or from distal end portions or tips of the elongate electrically conductive elements to expose conductive material. Removing the insulating layer may comprise laser ablation or mechanical removing using for example a blade or the like. Further, removing the insulating layer may comprise a chemical treatment.

Embodiments of the present invention thus provide an electrode structure that is biocompatible, has small dimensions and enables contacting a large number of neurons simultaneously.

### BRIEF DESCRIPTION OF THE DRAWINGS

Notwithstanding any other forms which may fall within the scope of the disclosure as set forth in the Summary, specific embodiments will now be described, by way of example only, with reference to the accompanying drawings in which:

FIG. 1(a) is a photograph of an electrode structure fabricated in accordance with an embodiment of the present invention;

FIG. 1(b) is a close-up photograph of an electrode structure fabricated in accordance with an embodiment of the present invention;

FIG. 2(a) is a schematic representation of an insulating substrate in accordance with an embodiment of the present invention;

FIG. 2(b) is another schematic representation of an insulating substrate in accordance with an embodiment of the present invention;

FIGS. 3 to 5 are schematic representations illustrating a method of fabricating an electrode structure in accordance with an embodiment of the present invention;

FIG. 6 is a flow chart of a method of fabricating an electrode structure in accordance with an embodiment of the present invention.

### DETAILED DESCRIPTION OF SPECIFIC EMBODIMENTS

Embodiments of the present invention relate to a method of fabricating an electrode structure that may be implanted into the human body for communication with the nervous system for example to treat or monitor a medical condition.

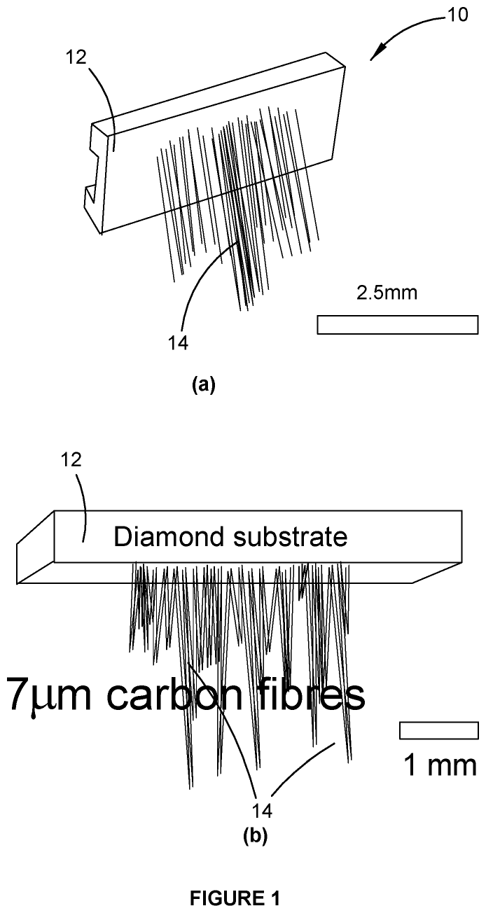

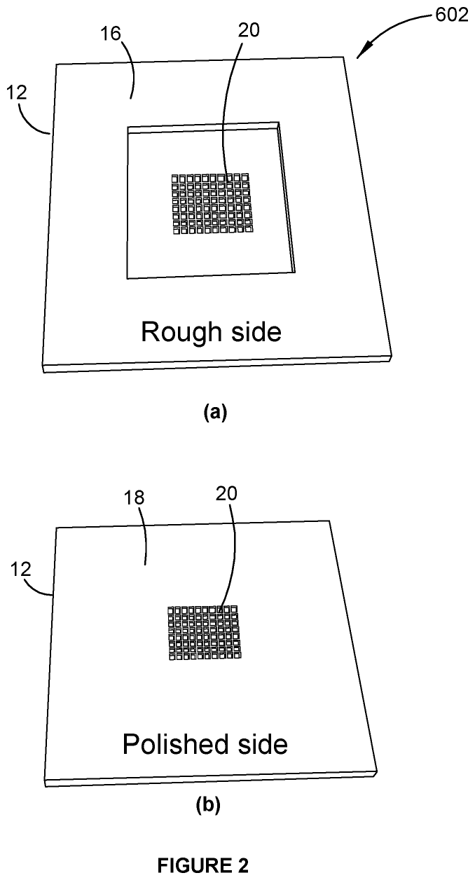

FIG. 1 illustrates an electrode structure 10 fabricated in accordance with an embodiment of the present invention. The electrode structure 10 comprises an electrically insulating substrate 12 and a plurality of elongate electrically conductive elements 14 protruding at a surface of the electrically insulating substrate 12. In this particular example, the electrode structure 10 comprises an array of approximately 400 elongate electrically conductive elements 14 protruding from the surface of the insulating substrate 12 (between 50 and 100 per square millimetre). The insulating substrate 12 comprises a diamond material such as poly-crystalline or single-crystalline diamond material. It is subsequently referred to as ‘diamond substrate’. Diamond material is chosen for its biocompatible properties as well as for being biochemically stable and having a very high thermal conductivity, which is beneficial for implanting into the human body. It is also highly electrically insulating, allowing for the electrical isolation of neighbouring electrodes even if they are very close to each other. It is envisaged that the insulating substrate may alternatively comprise a ceramic material having biocompatible properties such as alumina, sapphire, and/or silicon carbide or another suitable material.

Each elongate conductive element 14 is a carbon fibre element and in this specific example has a diameter of approximately 7 μm. However, it will be understood that the carbon fibre elements may have any diameter in the range of 2-20 μm or another suitable diameter.

Carbon material can be used for an electrochemical detection within the nervous system. It is however also envisaged that the elongate conductive elements 14 comprise alternatively metallic micro-wires.

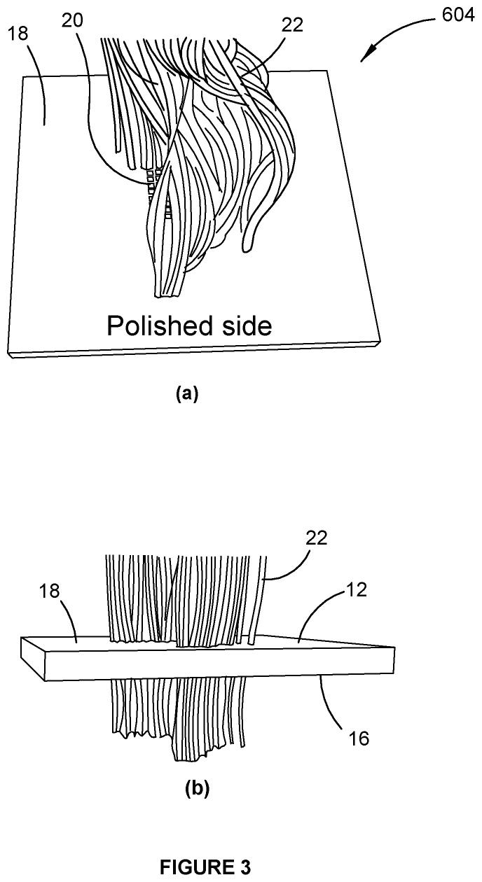

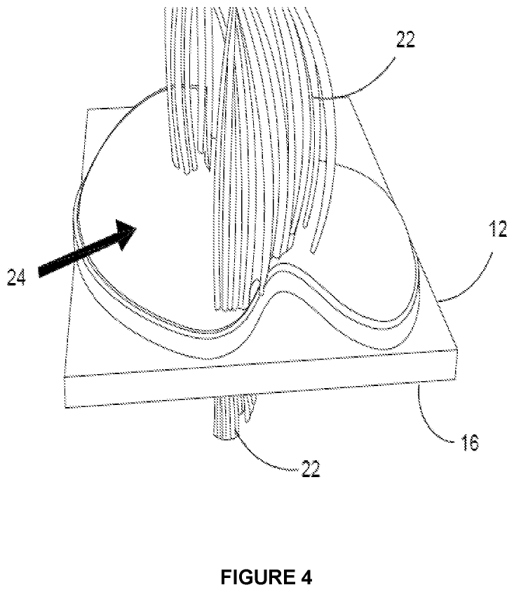

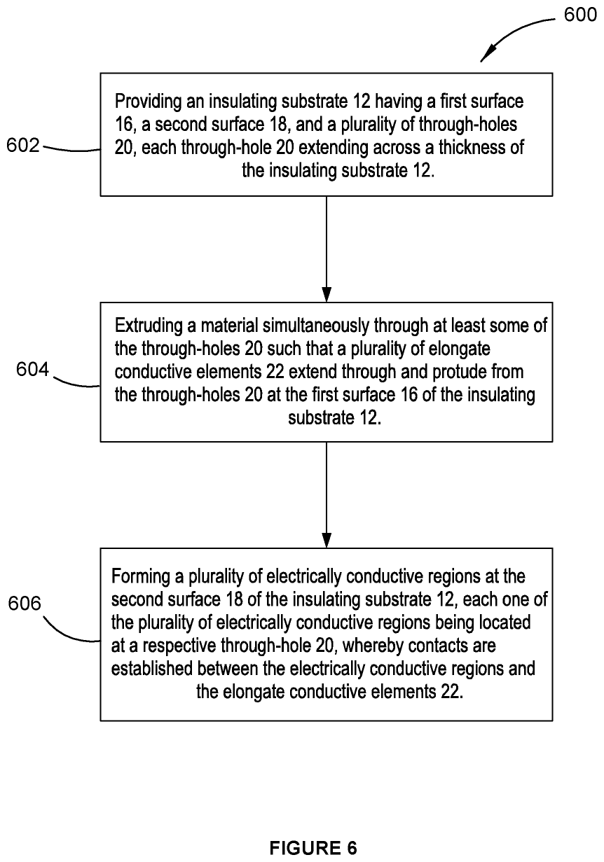

FIGS. 2-5 schematically illustrate processing steps of fabricating an electrode structure in accordance with a specific embodiment of the present invention, and FIG. 6 is a corresponding flow chart 600.

FIG. 2 illustrates more specifically a diamond substrate 12 provided in step 602 for fabricating the electrode structure 10. As can be seen on FIG. 2(a), the diamond substrate 12 has a first surface 16. FIG. 2(b) illustrates a second surface 18 of the diamond substrate 12 opposite the first surface 16. The diamond substrate also has a plurality of through-holes 20. Each through-hole 20 extends across a thickness of the diamond substrate 12. The through-holes 20 may for example be drilled into the diamond substrate 12 using a laser or a focused ion beam technique.

In a first embodiment of the present invention, the method of fabricating the electrode structure 10 comprises step 604 of extruding a solid material through at least some of the through-holes 20 of the diamond substrate 12.

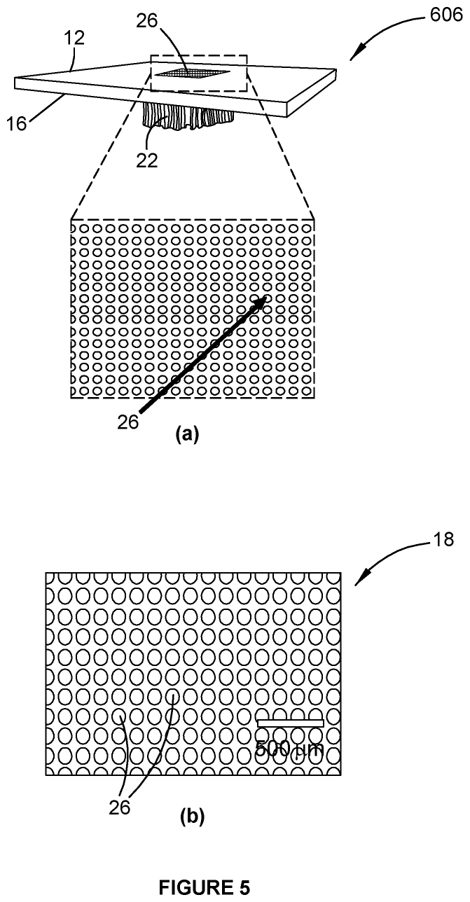

Specifically, as illustrated in FIG. 3, a bundle of carbon fibre elements 22 is positioned at the second surface 18 of the diamond substrate 12. The carbon fibre elements 22 are moved simultaneously or sequentially through the through-holes 20 using a press (not shown), such as a hydraulic press, and the through-holes 20 have a tapered cross-sectional shape such that each carbon fibre element 22 extends through and protrudes from a respective through-hole 20 of the first surface 16.

Each carbon fibre element 22 in the bundle has in this specific example a diameter of approximately 5-10, such as 7 μm. However, it will be understood that the carbon fibre elements 22 in the bundle may not all have the same diameter.

In the present example, the bundle comprises 100-1000 carbon fibres or rods 22 that are moved simultaneously or sequentially though the through-holes 20, and the carbon fibre elements 22 are extruded such that between 50 and 100 carbon fibre elements 22 protrude per square millimetre from the surface 16 of the electrically insulating substrate 12.

FIGS. 4 and 5 illustrate step 606 of the method of fabricating an electrode structure. FIG. 4 illustrates the step of applying an electrically conductive material such as an active brazing alloy paste 24 at the second surface 18 of the diamond substrate 12. The active brazing alloy paste 24 is heated to a suitable temperature (such as a temperature of approximately 900° C.) so that the active brazing alloy 24 melts and fills region in the though-holes 20 in order to secure and bond the carbon fibres 22 to the diamond substrate 12. The active brazing alloy paste 24 is then allowed to cool and the solidified active brazing alloy paste 24 is polished until it has a surface that is substantially flush with that of the diamond substrate 12. Electrically conductive regions 26 are thus formed at the second surface 18, and each electrically conductive region 26 is located at a respective through-hole 20, as is illustrated in FIG. 5(a) and more specifically in FIG. 5(b). The electrically conductive regions 26 are electrically coupled to the carbon fibres 22 and form hermetic seals. The electrically conductive regions 26 form an array of electrodes that can be used for bonding to an electronic component (not shown) such as, for example, a microprocessor, an application specific integrated circuit (ASIC), or other electronic components. The electrode structure 10 thus fabricated is any electrode array and comprises between 50 and 100 electrodes and respective carbon fibre elements 22 per square millimetre.

The electrically conductive material may alternatively be provided in any other suitable form. For example, a suitable soldering paste may be used in a manner similar to the active brazing material.

The carbon fibre elements 22 protruding from the first surface 16 of the diamond substrate 12, such as the elongate electrically conductive elements 14 illustrated in the photographs in FIG. 1, typically have a length between 1 and 10 mm, but maybe as short as 0.1 mm

The carbon fibres 22 are flexible and can be shaped to adapt to the human tissue into which the electrode structure is implanted. For example, an implant such as a retinal prosthesis needs to adapt to the curvature of the retina. The electrodes protruding from the electrically insulating substrate 12 can relatively easily adapt to the shape of the human tissue while being less invasive and minimising damage to the human tissue.

In another specific embodiment of the present invention, the method of fabricating the electrode structure 10 comprises step 604 of extruding a liquid material or a paste simultaneously or sequentially through the through-holes 20 of the diamond substrate 12.

The liquid material may be a conductive ink comprising carbon such as a graphene ink, or a conductive polymer composite such as Poly(3,4-ethylenedioxythiophene) (PEDOT).

The conductive ink is extruded simultaneously or sequentially through the through-holes 20 of the diamond substrate 12. When the conductive ink is fully extruded through the through-holes 20 of the diamond substrate 12 and has hardened, an array of solid elongate conductive elements is formed. The solid elongate electrically conductive elements 14 protrude from the through-holes 20 at the first surface 16 of the diamond substrate 12. Electrically conductive regions are then formed at the second surface 18 of the diamond substrate 12 in accordance with the step 606 illustrated in FIGS. 4 and 5 and described above.

The carbon fibres 22 or the electrically conductive elements that were formed by extruding conductive ink and protrude from of the diamond substrate 12 are then processed to electrically insulate them from each other and to improve a biocompatibility and/or electrochemical properties of the electrode structure 10. Electrically insulating layers are formed on the elongate electrically conductive elements 14, 22 using a vacuum deposition technique. The insulating layers comprise for example silicon dioxide and/or a poly(p-xylylene) polymer (parylene). The insulating layers can be selectively removed from the electrically conductive elements 14, 22 using for example a laser cutter. For example, portions of the insulating layers may be removed from side portions of some or all of the electrically conductive elements so that electrical contact with the electrically conducive elements can be established at predefined positions along the length of the electrically conductive elements. Alternatively or additionally, portions of the electrically insulating layer may be removed from distal end portions of the elongate electrically conductive elements.

Further, an electrically conductive layer may be formed using an electrochemical deposition technique onto a tip or distal end portion of each of the elongate electrically conductive elements 14, 22.

The electrically conductive layer may comprise a conductive polymer or a platinum group metal. Alternatively, an electrically conductive doped diamond material may be deposited onto the distal end-portions or tips of the elongate electrically conductive elements 14, 22.

Additionally or alternatively, the electrically conductive layer may comprise organic molecules suitable for electrochemically functionalising the surface of the distal end-portions or tips of the elongate electrically conductive elements 14, 22. For example, antibodies or enzymes may be deposited onto the tips of the elongate electrically conductive elements 14 22, using a diazotization technique.

The electrode structure 10 fabricated in accordance with embodiments of the present invention, once implanted in a human tissue such as brain tissue, is adapted for detecting or stimulating neural activity. An electrochemical functionalization of the surface of the tips of the elongate electrically conductive elements 14, 22, may for example contribute to establishing contact between the elongate electrically conductive elements 14 and the neurons. The selective removal of the electrically insulating layer from side portions of the elongate electrically conductive elements 14, 22 may for example enable stimulating neural activity at selected distances from the substrate 12 when the array of electrodes is implanted in a human tissue and bonded to an electronic component for detection and/or stimulation of the neural activity.

Further, the electrode structure 10 fabricated in accordance with embodiments of the present invention is a high density array of electrodes that is biocompatible, has small dimensions, and enables contacting a large number of neurons simultaneously. Insulating the elongate electrically conductive elements 14, 22 from each other ensures that each of the elongate electrically conductive elements 14, 22 of the electrode structure 10 can independently and simultaneously contribute to the detection or stimulation of a neural activity despite the small dimensions and high density of the electrode structure 10.

Modifications and variations as would be apparent to a skilled addressee are determined to be within the scope of the present invention.

1. A method of fabricating an electrode structure, the method comprising:
providing an electrically insulating substrate having a first surface, a second surface opposite the first surface, and a plurality of through-holes, each through-hole extending across a thickness of the insulating substrate;
extruding a material through at least some of the through-holes resulting in a plurality of elongate electrically conductive elements extending through and protruding from the through-holes at the first surface of the electrically insulating substrate; and
forming a plurality of electrically conductive regions at the second surface of the electrically insulating substrate, each electrically conductive region being located at a respective through-hole, whereby the electrically conductive regions are electrically coupled to the elongate electrically conductive elements.

2. The method of claim 1, wherein each elongate electrically conductive element extends through and protrudes from a respective through-hole.

3. The method of claim 1 wherein the step of extruding a material through at least some of the through-holes comprises extruding the material sequentially through at least some of the holes.

4. The method of claim 1 wherein the step of extruding a material through at least some of the through-holes comprises extruding the material simultaneously through at least some of the holes.

5. The method of claim 1, wherein the electrode structure is an array of electrodes.

6. The method of claim 5, wherein the array comprises more than 20, more than 50, more than 100 or more than 500 electrodes and respective elongate electrically conductive elements per square millimetre.

7. The method of claim 1, wherein the material that is extruded through at least some of the through-holes comprises carbon.

8. The method of claim 1, wherein extruding a material through at least some of the through-holes comprises moving the plurality of elongate electrically conductive elements through the through-holes.

9. (canceled)

10. The method of claim 8, wherein forming the plurality of electrically conductive regions at the second surface of the electrically insulating substrate comprises bonding the elongate electrically conductive elements to the substrate and the electrically conductive regions.

11. The method of claim 8, wherein the elongate electrically conductive elements are carbon fibres or rods.

12. (canceled)

13. The method of claim 8, wherein the plurality of elongate electrically conductive elements that are moved through the through-holes comprises at least 100, such as 100-500, 500-1000 or more than 1000 elongate electrically conductive elements.

14. The method of claim 1, wherein extruding the material comprises extruding a liquid material or a paste through at least some of the through-holes, the material being selected and extruded such that a plurality of solid elongate conductive elements are formed when the material has hardened, the electrically conductive elements protruding from the through-holes at the first surface of the electrically insulating substrate.

15. The method of claim 14, wherein the liquid material or paste comprises a conductive ink.

16. The method of claim 15, wherein the conductive ink comprises graphene.

17. The method of claim 15, wherein the conductive ink comprises a conductive polymer.

18. The method of claim 1, wherein at least some of the elongate electrically conductive elements have a substantially circular cross-sectional shape with a diameter in the range of 2-20 μm.

19. The method of claim 18, wherein at least some of the elongate electrically conductive elements have a diameter of 2-5 μm, 5-7 μm, 7-10 μm.

20. The method of claim 1, wherein the elongate electrically conductive elements protrude from the through-holes at the first surface of the electrically insulating substrate with a length of at least 0.01 mm, at least 0.1 mm, such as between 1 mm and 10 mm.

21. The method of claim 1, wherein each through-hole has a tapered cross-sectional shape.

22. (canceled)

23. (canceled)

24. (canceled)

25. The method of claim 1, wherein the electrically conductive regions are formed using a brazing alloy paste.

26-33. (canceled)
